(19) United States
(12) Patent Application Publication
(10) Pub. No.: US 2005/0261221 A1
(43) Pub. Date: Nov. 24, 2005

(54) Compositions and methods for enhanced delivery of compounds via transfection complexes

(76) Inventors: Li Wang, Evanston, IL (US); Robert C. MacDonald, Evanston, IL (US)

Correspondence Address: MEDLEN & CARROLL, LLP, Suite 350, 101 Howard Street, San Francisco, CA, 94105, US

(21) Appl. No.: 11/069,115

(22) Filed: Mar. 1, 2005

Related U.S. Application Data
(60) Provisional application No. 60/549,002, filed on Mar. 1, 2004.

(51) Int. Cl. A61K 48/00; C12N 15/85

(52) U.S. Cl. 514/44; 435/455

(57) ABSTRACT

The present invention relates to compositions and methods for enhanced delivery of compounds (e.g., drugs, nucleic acids, peptides, etc.) via transfection complexes (e.g., liposomes). In particular, the present invention utilizes agents that synergistically enhance the ability of a transfection complex to deliver a compound to cells or tissue.

The present invention claims priority to U.S. Provisional Patent Application Ser. No. 60/549,002, filed Mar. 1, 2004, the disclosure of which is herein incorporated by reference in its entirety.

The present invention was generated, in part, by support from NIH grant GM52329. The government may have certain rights in the invention.

### FIELD OF THE INVENTION

The present invention relates to compositions and methods for enhanced delivery of compounds (e.g., drugs, nucleic acids, peptides, etc.) via transfection complexes (e.g., liposomes). In particular, the present invention utilizes agents that synergistically enhance the ability of a transfection complex to deliver a compound to cells or tissue.

### BACKGROUND

Overcoming inefficient delivery of functional therapeutic genes into target cells is a major challenge in gene therapy of cancer, metabolic diseases and some infectious diseases. The use of viral vectors has several major drawbacks [1], including the risk of immunogenicity, potential recombination with endogenous viruses and cytotoxicity. These limitations have stimulated studies to improve methods of non-viral gene delivery. Among such non-viral vectors, DNA-cationic lipid complexes (“lipoplexes”) have been utilized for numerous in vitro and in vivo gene delivery applications [2-7]. Lipoplexes have the advantage of low immunogenicity, safety, ability to package large DNA molecules, and ease of preparation [8]. Although significant advances in cationic lipoplexes have led to significant improvements, lipid-mediated gene delivery has not yet approached the high efficiency of viruses. Improved compositions and methods are needed.

### DESCRIPTION OF THE DRAWINGS

FIG. 1. Effect of free colchicine on the extent of transfection of VSMCs as a function of the colchicine concentration, with and without 20% FBS. The cells were treated with colchicine for 3 h, washed with PBS, and then treated with DNA-lipid complexes for 3 h. “No serum” and “20% FBS” denote that serum was absent or present during the 3 h incubation with transfection complexes. The figure shows the increase in β-galactosidase activity expressed as fluorescence intensity relative to no pretreatment with colchicine and absence of serum during transfection treatment. The number above the last bar denotes the fold increase in the fluorescence intensity at 8 μg/ml colchicine relative to no pretreatment with colchicine but presence of 20% FBS during transfection treatment, i.e., in 20% FBS, 8 μg/ml colchicine led to a 24-fold increase in expression relative to no colchicine. Data represent the means±S.D. of quadruplicate wells in one typical experiment. The experiment was repeated four times with consistent results.

FIG. 2. Effect of free vinblastine on the extent of transfection of VSMCs as a function of the vinblastine concentration, with and without 20% FBS. The cells were treated with vinblastine for 3 h, washed with PBS, and then treated with DNA-lipid complexes for 3 h. “No serum” and “20% FBS” denote that serum was absent or present during the 3 h incubation with transfection complexes. The figure shows the increase in β-galactosidase activity expressed as fluorescence intensity relative to no pretreatment with vinblastine and absence of serum during transfection treatment. The number above the last bar denotes the fold increase in the fluorescence intensity at 2.5 μM vinblastine relative to no pretreatment with vinblastine but presence of 20% FBS during transfection treatment, i.e., in 20% FBS, 2.5 μM vinblastine led to a 12-fold increase in expression relative to no vinblastine. Data represent the means±S.D. of quadruplicate wells in one typical experiment. The experiment was repeated twice with consistent results.

FIG. 3. Effect of vinblastine on the extent of transfection of VSMCs, when the vinblastine was incorporated in the lipid prior to generation of liposomes and formation of lipoplexes, as a function of the vinblastine concentration, with and without 20% FBS. The cells were treated with DNA-lipid complexes for 3 h. “No serum” and “20% FBS” denote that serum was absent or present during the 3 h incubation with transfection complexes. The figure shows the increase in β-galactosidase activity expressed as fluorescence intensity relative to absence of vinblastine and serum during transfection treatment. The number above the last bar denotes the fold increase in the fluorescence intensity after the incorporation of 1% (w/w) vinblastine in the lipid relative to absence of vinblastine but presence of 20% FBS during transfection treatment, i.e., in 20% FBS, 1% (w/w) vinblastine led to a 30-fold increase in expression relative to no vinblastine. Data represent the means±S.D. of quadruplicate wells in one typical experiment. The experiment was repeated five times with consistent results.

FIG. 4. Taxol blocks the stimulation of transfection of VSMCs by colchicine. For pretreatment with taxol or colchicine alone, the cells were incubated with 4 μM taxol or 0.8 μg/ml colchicine for 3 h, washed with PBS, and then treated with DNA-lipid complexes for 3 h. For co-treatment with both taxol and colchicine, the cells were first incubated with 4 μM taxol for 30 min and then colchicine was added to the medium. “No serum” and “20% FBS” denote that serum was absent or present during the 3 h incubation with transfection complexes. The figure shows the increase in β-galactosidase activity expressed as fluorescence intensity relative to no pretreatment with drug and absence of serum during transfection treatment. Data represent the means±S.D. of quadruplicate wells in one typical experiment. The experiment was repeated twice with consistent results.

FIG. 5. Effect of vinblastine on the intracellular distribution of lipoplexes. Lysosomes were labeled with Rh-dextran (A and C) and the distribution of lipoplexes was made visible by incorporating DOPE-N-NBD into liposomes used for the lipoplexes (B and D). The cells were labeled with Rh-dextran overnight, then washed and incubated for 2 h in normal medium to chase the Rh-dextran into lysosomal compartments. Subsequently, the cells were incubated with DOPE-N-NBD-labeled normal lipoplexes (A and B) or VB-lipoplexes (C and D) for 3 h. After the incubation, the cells were treated with CellScrub buffer to remove surface-bound lipoplexes and were observed under fluorescence microscope (×100 objective). E is the overlap of A and B, and F is the overlap of C and D.

FIG. 6. Optimal effect of Con A, monensin and gentamicin on transfection of VSMCs. Cells were treated with 100 μg/ml Con A for 30 min, 1 μM monensin for 15 min, or 0.1 mg/ml gentamicin for 15 min, and washed with PBS, and then incubated with normal DNA-lipid complexes, or cells without any pretreatment were incubated with VB-lipoplexes for 3 h. After that fresh 20% FBS was added. The figure shows the fluorescence intensity relative to no pretreatment. Data represent the means±S.D. of quadruplicate wells in one typical experiment. The experiment was repeated twice with consistent results. *Compared with VB-lipoplexes, P<0.01.

FIG. 7. Involvement of NF-κB activation in the vinblastine effect, as indicated by the inhibition of the transfection of normal and VB-lipoplexes by PDTC (A) and TLCK (B) as well as by alteration of the cellular distribution of NF-κB (C, D and E). In A and B, the cells were treated with normal or VB-lipoplexes for 3 h and then washed, and fresh 20% FBS containing 10 μM PDTC (A) or 100 μM TLCK (B) was added. The figures show the fluorescence intensity, which is proportional to β-galactosidase expression. Black and gray columns denote normal lipoplexes and VB-lipoplexes, respectively. Experiments were performed in quadruplicate. The cells were incubated with culture medium (C), normal lipoplexes (D) or VB-lipoplexes (E) for 3 h, and then the immunofluorescence staining of NF-κB was done according to the procedure in MATERIALS AND METHODS. The experiment was repeated twice with consistent results.

### DEFINITIONS

To facilitate an understanding of the present invention, a number of terms and phrases are defined below:

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

The terms “test compound” and “candidate compound” refer to any chemical entity, pharmaceutical, drug, and the like that is a candidate for use to treat or prevent a disease, illness, sickness, or disorder of bodily function (e.g., cancer). Test compounds comprise both known and potential therapeutic compounds. A test compound can be determined to be therapeutic by screening using the screening methods of the present invention.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water and industrial samples. Such examples are not however to be construed as limiting the sample types applicable to the present invention.

As used herein, the terms “drug” and “chemotherapeutic agent” refer to pharmacologically active molecules that are used to diagnose, treat, or prevent diseases or pathological conditions in a physiological system (e.g., a subject, or in vivo, in vitro, or ex vivo cells, tissues, and organs). Drugs act by altering the physiology of a living organism, tissue, cell, or in vitro system to which the drug has been administered.

### DETAILED DESCRIPTION OF THE INVENTION

The present invention relates to compositions and methods for enhanced delivery of compounds (e.g., drugs, nucleic acids, peptides, etc.) via transfection complexes (e.g., liposomes). In particular, the present invention utilizes agents that synergistically enhance the ability of a transfection complex to deliver a compound to cells or tissue.

In some embodiments, the agent is improves vesicle trafficking (e.g., reduced the ability of a cell to deal with the transfection complex as a foreign agent). In some embodiments, the agent interferes with trafficking after endocytosis. In some preferred embodiments, the agent regulates microtubule or microfilament structure or function. In some embodiments, the agent provides a signal leading to increased expression (through direct or indirect mechanisms) of a delivered gene. In some embodiments, the agent controls the expression of a therapeutic gene (e.g., a stimulatory transcription factor).

The present invention is not limited by the nature of the transfection complex. In some embodiments, the transfection complex comprises natural or synthetic lipids (cationic lipids, neutral lipids, anionic lipids). In some embodiments, the transfection complex comprises a liposome. In some embodiments, the liposome comprises cationic lipids. Examples of materials that find use in the transfection complexes of the present invention include, but are not limited to, liposomes include, lecithin, lysolecithin, lysophosphatidylethanolamine, phosphatidylserine, phosphatidylinositol, sphinogomyelin, cephalin, cardiolipin, phosphatidic acid, cerebrosides, acetylphosphate, dioleoylphosphatidylcholine (DOPE), dipalmitoylphosphatidylcholine, dioleoylphosphatidylglycerol (DOPC), dipalmitoylphosphatidylglycerol, dioleoylphosphatidylethanolamine (DOPE), palmitoyloleoylphosphatidylcholine (POPC), palmitoyloleoylphosphatidylethanolamine (POPE), dioleoylphosphatidylethanolamine 4-(N-maleimidomethyl)cyclohexane-1-carboxylate (DOPE-MAL), diheptadecanoyl phosphatidylethanolamine, dilauroylphosphatilylethanolamine, dimyristoylphosphatidylethanolamine, distearoyl phosphatidylethanolamine, beta-linoleoyl-gammapalmitoyl phosphatidylethanolamine and beta-oleoyl-gammapalmitoyl phosphatidylethanolamine), 1,2-diolelyloxy-3-(trimethylamino)propane (DOTAP); N-1-(2,3,-ditetradecyloxy)propyl-N,N-dimethyl-N-hydroxyethylammonium bromide (DMRIE); N-1-(2,3,-dioleyloxy)propyl-N,N-dimethyl-N-hydroxy ethylammonium bromide (DORIE); N-1-(2,3-dioleyloxy)propyl-N,N,N-trimethylammonium chloride (DOTMA); 3β N-(N′,N′-dimethylaminoethane) carbamoly cholesterol (DC-Chol); and dimethyldioctadecylammonium (DDAB).

Experiments conducted during the development of the invention showed that microtubule-depolymerizing agents (e.g., colchicine, vinblastine (VB), vincristine (VC), nocodazole, podophyllotoxin) were found to increase dramatically the transfection of cationic phospholipid-DNA (CMV-β-gal) complexes on cultured vascular smooth muscle cells (VSMCs). Thus, the present invention provides enhanced transfection complexes that include at least one microtubule-depolymerizing agent. A wide variety of transfection complexes may be so enhanced. While the invention is illustrated below with certain cationic lipid complexes, the present invention is not so limited.

Experiments conducted during the development of the present invention demonstrated that pretreatment of cells with free colchicine before addition of lipoplexes increased transgene expression both in presence and absence of serum. Free vinblastine had similar effects; however, vinblastine was more effective (˜30-fold maximal stimulation) when incorporated into the lipoplexes. Under optimal conditions, vincristine, nocodazole and podophyllotoxin produced 25- and 39-, 31- and 14-, 26- and 14-fold increases in the absence and presence of serum, respectively. Taxol, which stabilizes microtubules, had no effect on transfection, but it blocked the positive effect of colchicine. Cytochalasin B, which inhibits microfilament polymerization, had no effect of transgene expression. By fluorescence microscopy, normal lipoplexes colocalized with lysosomes. In contrast, there was little if any of colocalization of VB-lipoplexes with lysosomes. Because depolymerization of microtubules induces NFκB-dependent gene expression, the effect of PDTC (pyrrolidinedithiocarbamate) and TLCK (Nα-p-tosyl-L-lysine chloromethyl ketone), inhibitors of NF-κB activation, were tested; inhibition of vinblastine stimulation of transfection was 85% and 66%, respectively. Also, immunofluorescence microscopy showed that vinblastine induced the translocation of NF-κB from the cytoplasm to the nucleus. It is concluded that microtubule-depolymerizing agents, especially when incorporated into lipoplexes, dramatically increase transfection of VSMCs, probably by two mechanisms (although an understanding of the mechanism is not necessary to practice the present invention and the present invention is not limited to any mechanism of action): (i) inhibition of transport of lipoplexes to lysosomes; and (ii) activation of transcription (via NF-κB). There have been some reports on the use of pharmaceutical agents to enhance gene expression, but generally these have involved separate application of drug and gene. The ability to deliver a drug and a gene in a single therapeutic formulation has significant clinical implications. Thus, the present invention provides important new clinical methods.

Endocytosis has been suggested as the main cell internalization pathway of DNA-cationic lipid complexes [9, 10] and the efficiency of this step has been proposed to be an important rate-limiting factor in transfection [11, 12]. Recent studies have concluded that early escape of the complexes from endosomal/lysosomal compartments is crucial for efficient gene transfer and expression [13-15]. Indeed, entry of the complexes into the lysosomal compartment has been suggested to cause massive DNA degradation and to prevent transfection. Chloroquine, which increases endosomal pH and prevents endosome-lysosome fusion and thus aids escape of the complexes from the endosomes, has been used to enhance cationic lipid-mediated transfection [16-18].

Microtubules, a component of the cytoskeleton, consist of tubulin and several associated proteins. In addition to their roles, together with microfilaments and intermediate filaments in determining cell shape, microtubules are involved in motility, movement of cell surface receptors, and chromosomal segregation during mitosis. In addition, intact microtubules are required for translocation of endosomes to lysosomes [19-22]. Effects of the microtubule-active agents on gene transfection and expression are still a matter of debate [15, 23-27].

Colchicine, vinblastine (VB), vincristine (VC), nocodazole and podophyllotoxin belong to a class of microtubule-depolymerizing agent that binds specifically to tubulin and inhibits its polymerization. In the present invention, these agents were found to substantially increase the transfection of cultured vascular smooth muscle cells (VSMCs) by DNA-cationic phospholipid complexes. Some possible mechanisms were investigated and it was concluded that in addition to disrupting transport of lipoplexes to lysosomes, microtubule-depolymerizing agents also stimulate transcription, probably through an NFκB-dependent pathway (although an understanding of the mechanism is not necessary to practice the present invention and the present invention is not limited to any mechanism of action).

Effect of Colchicine on Transfection

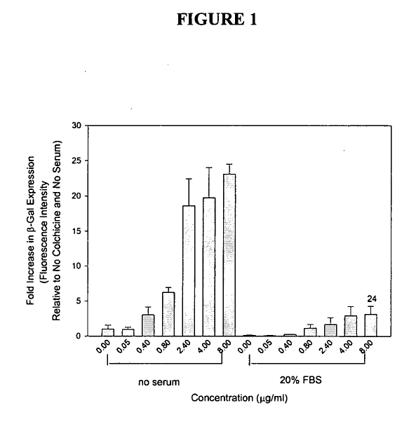

Pretreatment of cells with colchicine led to increased reporter gene expression, and this increase was colchicine concentration-dependent over the entire observed range (FIG. 1). Moreover, this stimulation was not influenced by serum. Transfection was increased ˜23-fold by pretreatment with 8.0 μg/ml colchicine both in the presence and absence of serum. When colchicine was added to cells simultaneously with lipoplexes, expression was not noticeably stimulated, except at 0.05 μg/ml colchicine and in the absence of serum, under which conditions transfection increased marginally.

Effect of Vinblastine on Transfection

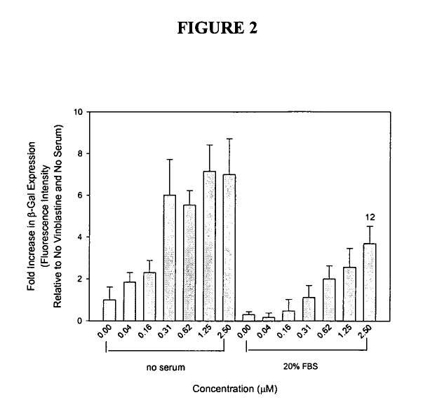

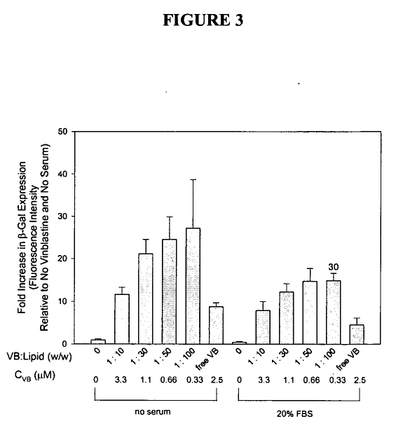

Similarly to colchicine, vinblastine pretreatment also increased transfection both in presence and absence of serum (FIG. 2). Since vinblastine is hydrophobic (soluble in chloroform, essentially insoluble in water), we also tested vinblastine that was incorporated in the lipid prior to generation of liposomes and formation of lipoplexes. This led to an additional increased transfection (FIG. 3). When 1% vinblastine was incorporated in the lipid, transfection increased ˜30-fold both in presence and absence of serum. Also, compared with a commercial non-viral transgene agent (Effectene), which is marketed by QIAGEN as specifically efficient in transfection of VSMCs, this vinblastine lipoplex formulation was an order of magnitude more effective. So this formulation was used in the experiments described below and is referred as VB-lipoplexes.

Effect of Vincristine, Nocodazole and Podophyllotoxin on Transfection

In order to investigate if the stimulation described above is specific to particular microtubule-depolymerizing agents, other agents were tested for an effect on transfection. As listed in Table 1, similar effects were observed.

Effect of Taxol on Transfection

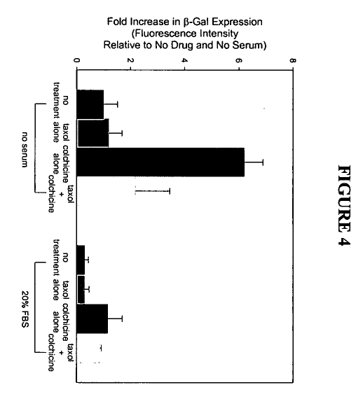

Taxol was tested because it stabilizes microtubules, an effect opposite to that of microtubule-depolymering agents. Pretreatment with taxol alone had no effect on transfection efficiency; however, taxol blocked the stimulating effect of colchicine that was added after taxol (FIG. 4). Similarly, pretreatment with taxol also blocked the stimulation of VB-lipoplexes.

Lack of Effect of Cytochalasin B on Transfection

Like microtubules, microfilaments are a component of the cytoskeleton, and are also involved in determining cell shape and motility. Cytochalasin B is an inhibitor of microfilament polymerization and its effect on transfection was examined. Cells were treated with 0.001-0.5 μM cytochalasin B for 3 h, washed with PBS, and then treated with DNA-lipid complexes for 3 h. No effect on transfection was observed.

X-Gal Histochemical Analysis and Cytotoxicity

X-gal staining and cytotoxicity determination were done. When tranfected with VB-lipoplexes, ˜15% and ˜5% cells were stained blue in the absence and presence of serum, respectively; in contrast, without vinblastine, less than 2% and 1% cells were stained in the absence and presence of serum, respectively. Moreover, the intensity of staining was subjected to image analysis (SigmaScan; SPSS Inc., Chicago, Ill.) and the cell staining intensity for VB-lipoplexes was ˜2-fold higher than for those treated with normal lipoplexes (65.5±13.5 vs 113.8±17.6. The intensity measured by SigmaScan is inversely related to absorbance. 10 random fields were measured, in which 270 cells were stained in case of VB-lipoplexes while only 49 cells were stained in case of normal lipoplexes). Compared to the absence of vinblastine, cell viability was 80 and 90% in the absence and presence of serum, respectively.

Fluorescence Microscopy

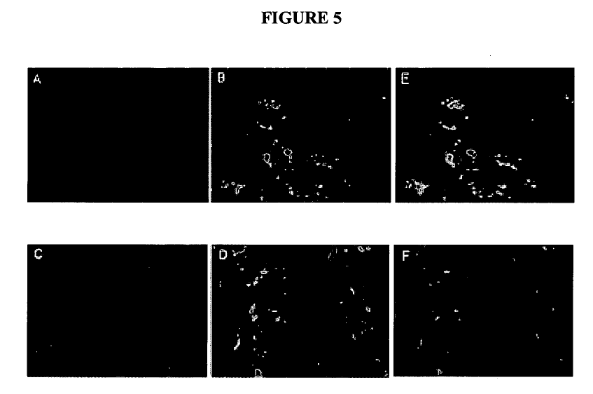

In order to investigate the contribution of microtubules to the transfection efficiency of VSMCs, we examined the effect of vinblastine on the intracellular distribution of lipoplexes by fluorescence microscopy. Lysosomes were labeled with Rh-dextran and the distribution of lipoplexes was made visible by incorporating DOPE-N-NBD into the liposomes used for the lipoplexes. Normal lipoplexes colocalized with Rh-dextran in lysosomes around the nuclei (FIGS. 5A, 5B and 5E). In contrast, in case of the VB-lipoplexes, there was little, if any overlap of the two fluorophores (FIG. 5F). Many lysosomal compartments contained no lipoplexes and a large proportion of the lipoplexes were distributed peripherally, probably in endosomes (FIGS. 5C and 5D). In addition, immunofluorescence microscopy with α-tubulin antibody revealed a marked disruption of microtubules in cells treated with VB-lipoplexes. The untreated cells were elongated with many microtubules visible paralleling to the long axis of the cell, whereas the treated cells were almost symmetrical and, although cytoplasmic staining was quite intense (but not entirely uniform), essentially no microtubules were large enough to be identifiable. These results further confirm the influence of vinblastine from VB-lipoplexes on microtubules.

Influence of Other Agents Known to Influence Trafficking Between Endosomes and Lysosomes

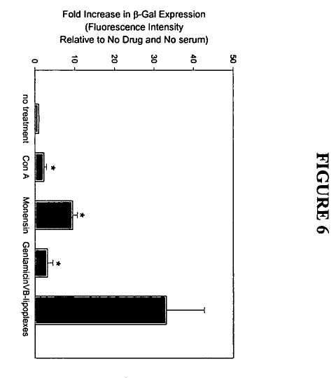

Other kinds of agents besides microtubule-active agents also influence trafficking between endosomes and lysosomes and they do so by different mechanisms. For example, concanavalin A (Con A) has been shown to inhibit endosome-lysosome fusion and thereby strongly suppresses the degradation of the exogenous endocytosed proteins [32]. Ionophores such as monensin elevate the lysosomal pH and thereby inhibit the degradation of endocytosed proteins [32]. Gentamicin induces phospholipidosis and as a result inhibits endosome-lysosome fusion [33, 34]. We therefore tested these agents to confirm the contribution of trafficking between endosomes and lysosomes to the transfection efficiency. Optimal transfection results were presented in FIG. 6. The largest effect (9.5-fold) was produced by monensin.

PDTC and TLCK

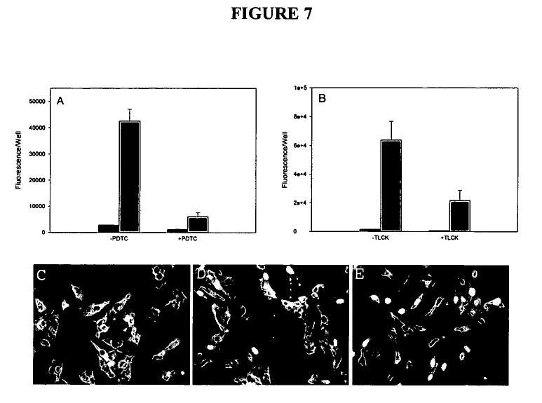

As indicated in the previous sections, other agents that influence trafficking between endosomes and lysosomes also increased transfection, but not as much as microtubule-depolymerizing agents. This suggests that the latter could be having effects in addition to those on vesicle trafficking (although an understanding of the mechanism is not necessary to practice the present invention and the present invention is not limited to any mechanism of action). One such possible effect is on transcription, for it has been reported that depolymerization of microtubules activates the sequence-specific transcription factor NF-κB and induces NFκB-dependent gene expression [35]. So we inquired whether microtubule-depolymerizing agents might have such transcription-related effects on the transfection of VSMCs. Two kinds of inhibitors of NF-κB activation were used to investigate this question: (1) PDTC, a thiol compound, which scavenges reactive oxygen intermediates, and thereby impedes the activation of NF-κB [36, 37]; (2) the serine protease inhibitor TLCK, which inhibits the proteolytic degradation of I-κB, an essential event in the activation of NF-κB [38]. As the data of FIG. 7 reveal, after treatment with PDTC, transfection of VSMCs with normal lipoplexes decreased by slightly over one half; however, that of VB-lipoplexes was reduced by 85%. Furthermore, the transfection stimulation by VB-lipoplexes was reduced from 15× to 5× by PDTC. Similarly, transfection of normal lipoplexes and VB-lipoplexes decreased 50% and 66%, respectively, after treatment with TLCK.

Immunocytochemistry of NF-κB

To further confirm the involvement of NF-κB activation in the vinblastine effect, immunofluorescence staining of NF-κB (p50) was done. One can see from FIGS. 7C, 7D and 7E that untreated cells or cells treated with normal lipoplexes shows primarily cytoplasmic localization with relatively little nuclear fluorescence. After treatment with VB-lipoplexes, large amounts of p50 were translocated to the nuclei, although significant amounts of p50 remained in the cytoplasm of some cells. To quantify this shift in p50 immunofluorescence, 1000 cells were counted. In the case of cells treated with normal lipoplexes, 5% of the nuclei were visibly labeled whereas in cells treated with VB-lipoplexes, 12% of the cells exhibited labeled nuclei.

There are several barriers to gene transfer by a cationic lipid. First, DNA-cationic lipid complexes must enter the cell, usually by endocytosis. Second, DNA must escape endosomes prior to their fusion with lysosomes. Third, DNA must enter the nucleus. Finally, DNA must be transcribed effectively and appropriately. Given that delivery of lipoplexes to lysosomes would lead to DNA degradation, it may be presumed that inhibition of endosomal translocation to lysosomes would promote transgene expression. Indeed, we found that all agents known to inhibit lysosome access to lipoplex DNA, by whatever mechanism, stimulated transgene expression. In addition, fluorescence microscopy (FIG. 5) confirmed the involvement of microtubules in intracellular dynamics of lipoplexes.

When vinblastine was incorporated into the lipid of lipoplexes, greater stimulation was observed than when it was used as free drug. This may be explained by more effective delivery of the drug by lipoplexes (although an understanding of the mechanism is not necessary to practice the present invention and the present invention is not limited to any mechanism of action). Furthermore, the effectiveness of the combination of vinblastine and DNA in a single delivery agent provides a considerable advantage with respect to clinical applications. There was no stimulation of expression when free colchicine and lipoplexes were added to the cell simultaneously. Microtubules are not only involved in the movement of endosomes to lysosomes, but also the formation of endocytic invaginations [20], so that endocytosis is also inhibited when microtubules are depolymerized. We assume inhibition of lipoplex uptake is responsible for decreased gene expression when lipoplexes and colchicine are simultaneously present (although an understanding of the mechanism is not necessary to practice the present invention and the present invention is not limited to any mechanism of action). However, if vinblastine is incorporated into the lipid, it cannot take effect until it is released from lipid. This, of course, also implies that there is little, if any free vinblastine in vinblastine/DNA lipoplexes (although an understanding of the mechanism is not necessary to practice the present invention and the present invention is not limited to any mechanism of action).

It has been reported [39] that (1) colchicine, nocodazole and vinblastine increase the level of nerve growth factor (NGF) mRNA, (2) taxol suppresses the effect of colchicine, and (3) the disruption of the microfilament network by cytochalasin B does not increase NGF mRNA. All of these observations are consistent with our results on the effects of microtubule-depolymerizing agents on transgene expression, so it is clear that transcription effects are likely to be involved in transfection by VB- and related lipoplexes (although an understanding of the mechanism is not necessary to practice the present invention and the present invention is not limited to any mechanism of action). Rosette et al. [35] found that microtubule depolymerization activates the sequence-specific transcription factor NF-κB and induces NFκB-dependent gene expression. Generally, the majority of NF-κB resides in the cytoplasm as a complex with its inhibitor IκB. Activation of NF-κB involves phosphorylation, dissociation and rapid proteolytic degradation of IκB, which allows the active p65/p50 subunit to migrate to the nucleus [38]. Also, reactive oxygen intermediates are important messengers in the activation of NF-κB [36, 37]. PDTC and TLCK are believed to block the NF-κB/IκB pathways through different mechanisms. PDTC scavenges reactive oxygen intermediates, whereas TLCK inhibits the proteolytic degradation of I-κB. In the experiments conducted during the development of the present invention, both agents were demonstrated to inhibit vinblastine stimulation of gene expression. X-gal histochemical analysis, which showed that the staining intensity of each stained cell was much stronger in the presence vinblastine than its absence, also confirms the transcriptional activation (although an understanding of the mechanism is not necessary to practice the present invention and the present invention is not limited to any mechanism of action). Immunofluorescence staining of NF-κB further showed that transcriptional regulation is involved in the effect of microtubule-depolymerizing agent on transgene expression in VSMCs. It is evident that there is some effect of PDTC and TLCK on expression even in absence of vinblastine; we presume this is because VSMCs themselves express a constitutive NF-κB-like activity [40] (although an understanding of the mechanism is not necessary to practice the present invention and the present invention is not limited to any mechanism of action).

There is evidence [41] that NF-κB not only regulates the expression of endogenous genes, but also of some exogenous genes. High multiplicity infection with a replication-deficient adenoviral vector activates NF-κB, which leads to augmented gene expression from the CMV-IEP (human cytomegalovirus immediate-early promoter). Since this was the promoter we used, the link between the endogenous NF-κB activation and exogenous gene increased expression may be the CMV-IEP element (although an understanding of the mechanism is not necessary to practice the present invention and the present invention is not limited to any mechanism of action). Experiments were conducted to replace CMV-IEP-β-Gal with SMGA-β-Gal (smooth muscle γ-actin gene promoter), which does not response to NF-κB. Transfection of this plasmid was not stimulated by vinblastine. This preliminary result supports an involvement of NF-κB but not of intracellular transport in the effect of vinblastine (although an understanding of the mechanism is not necessary to practice the present invention and the present invention is not limited to any mechanism of action).

Although in the past, liposomes have been formulated with drugs or genes in conjunction with molecules to assist release from endosomes or to increase transcription [42, 43], gene-drug combination in one formulation has not been extensively explored. As the results presented here reveal, such dual-acting liposomes are extremely effective.

Materials and Methods

Materials

Colchicine, vinblastine, vincristine, nocodazole, podophyllotoxin, taxol, cytochalasin B, X-gal, MTT (3-(4,5-dimethylthiazol-2-yl)-2,5-diphenyl tetrazolium bromide), Rh-dextran, concanavalin A, monensin, gentamicin, PDTC (pyrrolidinedithiocarbamate) and TLCK (Nα-p-tosyl-L-lysine chloromethyl ketone) were purchased from Sigma (St. Louis, Mo.). DOPE-N-NBD (1,2-dioleoyl-sn-glycero-3-phosphoethanolamine-N-(7-nitro-2-1,3-benzoxadiazol-4-yl) was purchased from Avanti Polar-Lipids, Inc (Alabaster, Ala.). CellScrub buffer was purchased from Gene Therapy Systems, Inc (San Diego, Calif.). EDOPC (1,2-dioleoyl-sn-glycero-3-ethylphosphocholine) was synthesized according to [28]. A β-galactosidase plasmid was purchased from Clontech Labtories Inc. (Palo Alto, Calif.) and propagated and purified by Bayou Biolabs (Harahan, La.). Rabbit anti-p50 NF-κB antibody and rabbit α tubulin antibody were obtained from Santa Cruz Biotechnology, Inc (Santa Cruz, Calif.) and Alexa Fluor 488 labeled goat anti-rabbit IgG was from Molecular Probes (Eugene, Oreg.), respectively.

Cell Culture

Primary vascular smooth muscle cells were obtained by removal of the thoracoabdominal aortas of Wistar rats, which were then stripped of endothelium and adventitia [29]. Medial VSMCs were maintained in Dubecco's Modified Eagle's Medium (DMEM), 20% fetal bovine serum (FBS), 200 units/ml penicillin, 200 μg/ml streptomycin (all Gibco, Gaitherburg, Md.) at 37° C. with 5% CO₂. At confluence, the cells were passaged using 0.25 mg/ml Trypsin/EDTA (Clonetics, Walkersville, Md.) and were used at passage 3-10 for these experiments.

Transfection

The cells were seeded in 96-well plates at 24 h before transfection at densities to give about 80% confluence at the time of transfection. Normally, EDOPC was suspended in D-PBS at 1 mg/ml to form liposomes. Liposomes and plasmid DNA were diluted in serum-free cell culture medium to 60 μg/ml for lipid and to 20 μg/ml for DNA, and liposomes were pipetted into an equal volume of plasmid DNA solution at a 3:1 weight ratio and mixed gently. The resultant DNA-lipid complexes were incubated at room temperature for 15 min and then added to the cells that were either in medium lacking serum or medium containing 20% fetal bovine serum (FBS). At 3 h after addition of DNA-lipid complexes, except as stated otherwise, the cells were washed with PBS and fresh medium containing 20% FBS was added. Generally, the cells were incubated with free drugs in 20% FBS DMEM for the indicated duration, and then, unless otherwise described, the cells were washed with PBS and incubated with DNA-lipid complexes. For experiments involving co-treatment with taxol and colchicine, the cells were first incubated with taxol for 30 min and then colchicine was added to the medium. For experiments in which vinblastine was incorporated in the lipid, the indicated amount of vinblastine was mixed with EDOPC in chloroform and the chloroform was removed under an N₂ stream and high vacuum. Subsequent steps were same as for the normal lipoplexes.

Cells were assayed for β-galactosidase activity 24 h after transfection with a microplate fluorometric assay [30], modified by inclusion of a heating step (50° C., 45 min) to inactivate endogenous enzyme activity. Following aspiration of the medium from each well, the cells were washed once with PBS and then lysed by addition of 100 μl lysis buffer (0.03% Triton X-100 in 100 mM HEPES, pH 7.8, containing 1 mM MgSO₄, 10 mM KCl). The plates were placed at 50° C. for 45 min and then allowed to cool to room temperature. 10 μl 100 μM FDG (fluorescein di-β-D-galactopyranoside) was added into each well. Fluorescence was measured with a microplate fluorimeter (Model 7620, Cambridge Technology Inc.) after incubation at 37° C. for 3 h. Fluorescence intensity is proportional to β-galactosidase expression; 2000 fluorescence units corresponded approximately to 0.1 milliunit of β-galactosidase. The expression of VSMCs transfected with normal lipoplexes in absence of serum and any pretreatment was ˜0.1 milliunit per well.

X-gal staining was used to determine the number of transfected cells histochemically according to the procedure provided by Invitrogen Life Technologies (Carlsbad, Calif.). Viability of cells after transfection was assessed with the MTT method [31].

Fluorescence Microscopy

The cells were labeled with Rh-dextran (10 mg/ml) overnight, then washed and incubated for 2 h in normal medium to chase the Rh-dextran into lysosomal compartments. Subsequently, the cells were incubated with DOPE-N-NBD-labeled normal lipoplexes or VB-lipoplexes for 3 h. After incubation, the cells were treated with CellScrub buffer to remove most surface-bound lipoplexes. The distribution of lipoplexes was visualized under the fluorescence microscope (Leica DMIRE2).

Immunocytochemistry

Of NF-κB [44]

After treatment as for transfection, cells were fixed in 4% formaldehyde for 20 min, permeabilized with 0.1% Triton X-100 for 5 min, treated with 1% BSA for 30 min at 37° C. to block nonspecific antigenic sites, and treated for 1 h with a rabbit anti-p50 NF-κB antibody (1:20) at room temperature. The cells were then washed and incubated with an Alexa Fluor 488 labeled goat anti-rabbit IgG (10 μg/ml) for 1 h at room temperature. The cells were then observed under the fluorescence microscope.

Of Tubulin [45]

This procedure was similar to that described above except that a rabbit α tubulin antibody was used.

### REFERENCES

1. Treco, D. A., and Selden, R. F. (1995). Non-viral gene therapy. Mol. Med. Today. 1:314-321.
2. Felgner, P. L., et al. (1997). Nomenclature for synthetic gene delivery systems. Hum. Gene Ther. 8:511-512.
3. Felgner, P. L., et al. (1995). Improved cationic lipid formulations for in vivo gene therapy. Ann. N.Y. Acad. Sci. 772:126-139.
4. Morgan, R. A. and Anderson, W. F. (1993). Human gene therapy. Annu. Rev. Biochem. 62:191-217.
5. Byk, G., et al. (1998). Synthesis, activity, and structure—activity relationship studies of novel cationic lipids for DNA transfer. J. Med. Chem. 41:229-235.
6. Lee, E. R., et al. (1996). Detailed analysis of structures and formulations of cationic lipids for efficient gene transfer to the lung. Hum. Gene Ther. 7:1701-1717.
7. Coonrod, A., Li, F. Q., and Horwitz, M. (1997). On the mechanism of DNA transfection: efficient gene transfer without viruses. Gene Ther. 4:1313-1321.
8. Zabner, J. (1997). Cationic lipids used in gene transfer. Adv. Drug Deliv. Rev. 27:17-28.
9. Zabner, J., Fasbender, A. J., Moninger, T., Poellinger, K. A., and Welsh, M. J. (1995). Cellular and molecular barriers to gene transfer by a cationic lipid. J Biol. Chem. 270:18997-19007.
10. El Ouahabi, A., et al. (1999). Intracellular visualization of BrdU-labeled plasmid DNA/cationic liposome complexes. J Histochem. Cytochem. 47:1159-1166.
11. Wrobel, I. and Collins, D. (1995). Fusion of cationic liposomes with mammalian cells occurs after endocytosis. Biochim. Biophys. Acta. 1235:296-304.
12. Hui, S. W., Langner, M., Zhao, Y. L., Ross, P., Hurley, E., and Chan, K. (1996). The role of helper lipids in cationic liposome-mediated gene transfer. Biophys. J. 71:590-599.
13. Zhou, X. and Huang, L. (1994). DNA transfection mediated by cationic liposomes containing lipopolylysine: characterization and mechanism of action. Biochim. Biophys. Acta. 1189:195-203.
14. El Ouahabi, A., Thiry, M., Pector, V., Fuks, R., Ruysschaert, J. M., and Vandenbranden, M. (1997) The role of endosome destabilizing activity in the gene transfer process mediated by cationic lipids. FEBS Lett. 414:187-192.
15. Hasegawa, S., Hirashima, N., and Nakanishi, M. (2001). Microtubule involvement in the intracellular dynamics for gene transfection mediated by cationic liposomes. Gene Ther. 8:1669-1673.
16. Baru, M., Axelrod, J. H., and Nur, I. (1995). Liposome-encapsulated DNA-mediated gene transfer and synthesis of human factor IX in mice. Gene. 161:143-150.
17. Erbacher, P., Roche, A. C., Monsigny, M., and Midoux, P. (1996). Putative role of chloroquine in gene transfer into a human hepatoma cell line by DNA/lactosylated polylysine complexes. Exp. Cell Res. 225:186-194.
18. Otsuka, M., Baru, M., Delriviere, L., Talpe, S., Nur, I., and Gianello, P. (2000). In vivo liver-directed gene transfer in rats and pigs with large anionic multilamellar liposomes: routes of administration and effects of surgical manipulations on transfection efficiency. J Drug Target. 8:267-279.
19. Matteoni, R. and Kreis, T. E. (1987). Translocation and clustering of endosomes and lysosomes depends on microtubules. J Cell Biol. 105:1253-1265.
20. Elkjaer, M. L., Bim, H., Agre, P., Christensen, E. I., and Nielsen, S. (1995). Effects of microtubule disruption on endocytosis, membrane recycling and polarized distribution of Aquaporin-1 and gp330 in proximal tubule cells. Eur. J. Cell Biol. 67:57-72.
21. Thatte, H. S., Bridges, K. R., and Golan, D. E. (1994). Microtubule inhibitors differentially affect translational movement, cell surface expression, and endocytosis of transferrin receptors in K562 cells. J Cell Physiol. 160:345-357.
22. Liu, S. M., Magnusson, K. E., and Sundqvist, T. (1993). Microtubules are involved in transport of macromolecules by vesicles in cultured bovine aortic endothelial cells. J Cell Physiol. 156:311-316.
23. Lindberg, J., Fernandez, M. A., Ropp, J. D., and Hamm-Alvarez, S. F. (2001). Nocodazole treatment of CV-1 cells enhances nuclear/perinuclear accumulation of lipid-DNA complexes and increases gene expression. Pharm. Res. 18:246-249.
24. Chowdhury, N. R., et al. (1996). Microtubular disruption prolongs the expression of human bilirubin-uridinediphosphoglucuronate-glucuronosyltransferase-1 gene transferred into Gunn rat livers. J Biol. Chem. 271:2341-2346.
25. Santell, L., Marotti, K., Bartfeld, N. S., Baynham, P., and Levin, E. G. (1992). Disruption of microtubules inhibits the stimulation of tissue plasminogen activator expression and promotes plasminogen activator inhibitor type 1 expression in human endothelial cells. Exp. Cell Res. 201:358-365.
26. Blum, J. L. and Wicha, M. S. (1988). Role of the cytoskeleton in laminin induced mammary gene expression. J Cell Physiol. 135:13-22.
27. Nair, R. R., Rodgers, J. R., and Schwarz, L. A. (2002). Enhancement of transgene expression by combining glucocorticoids and anti-mitotic agents during transient transfection using DNA-cationic liposomes. Mol. Ther. 5:455-462.
28. MacDonald, R. C., Rakhmanova, V. A., Choi, K. L., Rosenzweig, H. S., and Lahiri, M. K. (1999). O-ethylphosphatidylcholine: A metabolizable cationic phospholipid which is a serum-compatible DNA transfection agent. J Pharm. Sci. 88:896-904.
29. Crowley, S. T., Dempsey, E. C., Horwitz, K. B., and Horwitz, L. D. (1994). Platelet-induced vascular smooth muscle cell proliferation is modulated by the growth amplification factors serotonin and adenosine diphosphate. Circulation. 90:1908-1918.
30. Rakhmanova, V. A. and MacDonald, R. C. (1998). A microplate fluorimetric assay for transfection of the beta-galactosidase reporter gene. Anal. Biochem. 257:234-237.
31. Mosmann, T. (1983). Rapid calorimetric assay for cellular growth and survival: application to proliferation and cytotoxicity assays. J Immunol. Methods. 65:55-63.
32. Seglen, P. O. (1983). Inhibitors of lysosomal function. Methods Enzymol. 96:737-764.
33. Reasor, M. J. and Kacew, S. (2001). Drug-induced phospholipidosis: are there functional consequences? Exp. Biol Med. (Maywood.). 226:825-830.
34. Van Bambeke, F., Montenez, J. P., Piret, J., Tulkens, P. M., Courtoy, P. J., and Mingeot-Leclercq, M. P. (1996). Interaction of the macrolide azithromycin with phospholipids. I. Inhibition of lysosomal phospholipase A1 activity. Eur. J. Pharmacol. 314:203-214.
35. Rosette, C. and Karin, M. (1995). Cytoskeletal control of gene expression: depolymerization of microtubules activates NF-kappa B. J. Cell Biol. 128:1111-1119.
36. Schreck, R., Rieber, P., and Baeuerle, P. A. (1991). Reactive oxygen intermediates as apparently widely used messengers in the activation of the NF-kappa B transcription factor and HIV-1. EMBO J. 10:2247-2258.
37. Fan, C., Li, Q., Ross, D., and Engelhardt, J. F. (2003). Tyrosine Phosphorylation of Ikappa Balpha Activates NFkappa B through a Redox-regulated and c-Src-dependent Mechanism Following Hypoxia/Reoxygenation. J Biol. Chem. 278:2072-2080.
38. Henkel, T., Machleidt, T., Alkalay, I., Kronke, M., Ben Neriah, Y., and Baeuerle, P. A. (1993). Rapid proteolysis of I kappa B-alpha is necessary for activation of transcription factor NF-kappa B. Nature. 365:182-185.
39. Baudet, C., Naveilhan, P., Jehan, F., Brachet, P., and Wion, D. (1995). Expression of the nerve growth factor gene is controlled by the microtubule network. J. Neurosci. Res. 41:462-470.
40. Lawrence, R., Chang, L. J., Siebenlist, U., Bressler, P., and Sonenshein, G. E. (1994). Vascular smooth muscle cells express a constitutive NF-kappa B-like activity. J Biol. Chem. 269:28913-28918.
41. Clesham, G. J., Adam, P. J., Proudfoot, D., Flynn, P. D., Efstathiou, S., and Weissberg, P. L. (1998). High adenoviral loads stimulate NF kappaB-dependent gene expression in human vascular smooth muscle cells. Gene Ther. 5:174-180.
42. Nishikawa, M, Kuramoto, T, Okabe, T, Takakura, Y, and Hashida M. (2003). Utilization of biological response against nonviral vectors for enhancing transgene expression. Mol. Ther. 7: S167.
43. Vereecque, R, Saudemont, A, Depil, S, and Quesnel, B. (2003). Chemotherapy increases transgene expression in leukemic cells. J Gene Med. Article available online in advance of print. Published online: 6 May 2003. Online ISSN: 1521-2254.
44. Ding, G. J. F., et al. (1998). Characterization and quantitation of NF-kappa B nuclear translocation induced by interleukin-1 and tumor necrosis factor-alpha—Development and use of a high capacity fluorescence cytometric system. J Bio Chem, 273: 28897-28905.

45. Spom L. A., Foster T. H. (1992). Photofrin and light induces microtubule depolymerization in cultured human endothelial cells. Cancer Res, 52: 3443-3448.

TABLE 1
Effect of vincristine, nocodazole and podophyllotoxin on transfection of
VSMCs

| | | | Fold increase of transfection |
|---|---|---|---|
| Optimal conditions | | | means ± S.D. |
| Vincristine | No serum | 1/50 | 25 ± 5.7*^{#} |
| | | (w/w) VC/ | |
| | | lipid | |
| | 20% FBS | 1/50 | 39 ± 6.9*^{#} |
| | | (w/w) VC/ | |
| | | lipid | |
| Nocodazole | No serum | 50 μM | 31 ± 5.0*^{#} |
| | | free drug | |
| | 20% FBS | 50 μM | 14 ± 2.0*^{##} |
| | | free drug | |
| podophyllotoxin | No serum | 20 μM | 26 ± 4.5*^{#} |
| | | free drug | |
| | 20% FBS | 20 μM | 14 ± 5.8**^{##} |
| | | free drug | |
| Vincristine was incorporated into the lipid mixture for the liposomes used to prepare the lipoplexes. The cells were treated with the DNA-lipid complexes for 3 h. For nocodazole and podophyllotoxin, the cells were treated with free drug for 3 h, washed with PBS, and then treated with DNA-lipid complexes for 3 h. “No serum” and “20% FBS” denote that serum was absent or present during the 3 h incubation with transfection complexes. Statistical significance of difference between various | | | |
| # treatments are as follows: | | | |

*Compared with no treatment, P < 0.001.
**Compared with no treatment, P < 0.01.
^{#}Compared with VB-lipoplexes, P > 0.01.
^{##}Compared with VB-lipoplexes, P < 0.01.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the art are intended to be within the scope of the following claims.

1. A composition comprising a transfection reagent, said transfection reagent comprising an agent that a) interferes with the elimination of the transfection reagent by a cell; or b) initiates a signal transduction event in a cell that increases the activity of a compound delivered by said transfection reagent.

2. The composition of claim 1, wherein said agent comprises a microtubule-depolymerizing agent.

3. The composition of claim 1, further comprising a therapeutic compound.

4. The composition of claim 3, wherein said compound comprises a nucleic acid.

5. The composition of claim 3, wherein said compound comprises a drug.

6. The composition of claim 1, further comprising a nucleic acid molecule and a drug.

7. The composition of claim 4, wherein said nucleic acid molecule comprises an expression vector.

8. The composition of claim 4, wherein said nucleic acid molecule comprises an small interfering RNA.

9. The composition of claim 4, wherein said nucleic acid molecule comprises an antisense oligonucleotide.

10. A method for transfecting a cell, comprising the step of exposing a cell to the composition of claim 1.

11. The method of claim 10, wherein said cell resides in vitro.

12. The method of claim 10, wherein said cell resides in vivo.

13. The method of claim 10, wherein said cell is prepared ex vivo.
